(19) United States
(12) Patent Application Publication
(10) Pub. No.: US 2007/0179367 A1
(43) Pub. Date: Aug. 2, 2007

(54) Method and Apparatus for Noninvasively Estimating a Property of an Animal Body Analyte from Spectral Data

(76) Inventors: Timothy L. Ruchti, Gilbert, AZ (US); Thomas B. Blank, Gilbert, AZ (US); Alexander D. Lorenz, Chandler, AZ (US)

Correspondence Address: GLENN PATENT GROUP, 3475 EDISON WAY, SUITE L, MENLO PARK, CA, 94025, US

(21) Appl. No.: 11/564,741

(22) Filed: Nov. 29, 2006

Related U.S. Application Data
(63) Continuation-in-part of application No. 10/849,422, filed on May 18, 2004.
Application No. 10/849,422 is a continuation-in-part of application No. 10/170,921, filed on Jun. 12, 2002, now Pat. No. 7,206,623.
Application No. 10/170,921 is a continuation-in-part of application No. 09/563,782, filed on May 2, 2000, now Pat. No. 6,415,167.
(60) Provisional application No. 60/741,333, filed on Nov. 30, 2005.

(51) Int. Cl. A61B 5/00

(52) U.S. Cl. 600/310; 600/316

(57) ABSTRACT

A method and apparatus for calibration development using clustering is disclosed. More particularly, the invention relates to subsequent calibration development using clusters that are individually interference compensated and to subsequent estimation. Estimation of analyte property values from data, such as noninvasive spectra, is improved by a calibration method that uses clusters that are individually interference-compensated.

### CROSS-REFERENCE TO RELATED APPLICATIONS

This application:

claims the benefit of U.S. provisional patent application Ser. No. 60/741,333 filed Nov. 30, 2005, which hereby is incorporated herein in its entirety by reference thereto; and

is a continuation-in-part of U.S. patent application Ser. No. 10/849,422, filed May 18, 2004, which is a continuation-in-part of U.S. patent application Ser. No. 10/170,921, filed Jun. 12, 2002, which is a continuation-in-part of U.S. Pat. No. 6,415,167 granted Jul. 2, 2002 (application Ser. No. 09/563,782, filed May 2, 2000), all of which are hereby incorporated herein in their entirety by this reference thereto.

### BACKGROUND OF THE INVENTION

1. Field of the Invention

The invention relates to calibration development and use in spectroscopy, and more particularly to calibration techniques for noninvasively estimating an analyte property from spectral data.

2. Description of the Related Art

Calibration typically is required in the field of tissue spectroscopy. Spectroscopy based noninvasive analyzers deliver external energy in the form of light to a specific sample site, region, or volume of the human body where the photons interact with a tissue sample, thus probing chemical and physical features. A number of incident photons are specularly reflected, diffusely reflected, scattered, and/or transmitted out of the body where they are detected.

Noninvasive Analyte Concentration Determination

A major difficulty in the noninvasive measurement of biological constituents of the body and analytes in tissue arises from the fact that many constituents, such as glucose, are present in very small concentrations compared to sources of interference. In particular, the complex, heterogeneous, and dynamic composition of the skin, together with profound variation over time, between tissue sample sites, such as within a patient and from patient-to-patient, interferes with and thereby attenuates the analyte signal of many target analytes, such as glucose. In addition, the actual tissue volume sampled and the effective or average pathlength of light is variable over time for a given subject and is variable between subjects. Therefore, optical properties of a tissue sample are modified in a highly nonlinear and profound manner and/or have an linear filter effect that introduces significant interference into noninvasive tissue measurements.

Glucose

Optical based glucose concentration analyzers typically use calibration. This is true for all types of glucose concentration analyzers such as traditional invasive, alternative invasive, noninvasive, and implantable analyzers. A fundamental feature of noninvasive glucose concentration analyzers is that they are secondary in nature, that is, they do not measure blood glucose concentrations directly. Therefore, a primary method typically is used to calibrate these devices to measure blood glucose concentrations properly.

Skin Structure

The structure and composition of skin varies widely among individuals. In addition, skin properties vary at different sites and over time on the same individual at the same site. The outer layers of skin include a thin layer known as the stratum corneum, a stratified cellular epidermis, and an underlying dermis of connective tissue. Below the dermis is the subcutaneous fatty layer or adipose tissue. The epidermis is the thin outer layer that provides a barrier to infection and loss of moisture, while the dermis is the thick inner layer that provides mechanical strength and elasticity. The epidermis layer is 10 to 150 μm thick and is divided into three layers, the basal, middle, and superficial layers. The basal layer borders the dermis and contains pigment-forming melanocyte cells, keratinocyte cells, langherhan cells, and merkel cells. In humans, the thickness of the dermis ranges from 0.5 mm over the eyelid to 4 mm on the back and averages approximately 1.2 mm over most of the body.

In the dermis, water accounts for approximately seventy percent of the volume of the dermis. The next most abundant constituent is collagen, a fibrous protein comprising seventy to seventy-five percent of the dry weight of the dermis. Elastin fibers, also a protein, are plentiful though they constitute only a small proportion of the bulk. In addition, the dermis contains a wide variety of structures, such as sweat glands, hair follicles, blood vessels, and other cellular constituents. Conversely, the subcutaneous layer, adipose tissue, is by volume approximately ten percent water and includes primarily cells rich in triglycerides and/or fat. The concentration of glucose varies in each layer according to the water content, the relative sizes of the fluid compartments, the distribution of capillaries, and the perfusion of blood. Due to the high concentration of hydrophobic fat and the high solubility of glucose in water, the average concentration of glucose in subcutaneous tissue is significantly lower than the glucose concentration in the dermis.

Optical Properties of Skin

When near-infrared light is delivered to the skin, a percentage of the incident radiation is reflected while the remainder penetrates into the skin. The proportion of reflected light, specular reflectance, is typically between four to seven percent of the delivered light over the entire spectrum from 250 to 3000 nm, for a perpendicular angle of incidence. The 93 to 96 percent of the incident light that enters the skin is attenuated due to absorption or scattering within the many layers of the skin. These two processes taken together essentially determine the penetration of light into skin, the tissue volume that is sampled by the light, and the transmitted or remitted light that is scattered from the skin. Diffuse reflectance or remittance is defined as that fraction of incident optical radiation that is returned from a turbid sample. Alternately, diffuse transmittance is the fraction of incident optical radiation which is transmitted through a turbid sample.

Light penetrating into the skin is transmitted, absorbed, and/or scattered. Absorption by various skin constituents account for the spectral extinction of the light within each layer. Scattering is the process by which photons are redirected to the skin surface to contribute to the observed diffuse reflectance of the skin.

Absorbance of light from 1100 to 2500 nm in tissue is primarily due to three fundamental constituents: water, protein, and fat. As the main constituent, water dominates the near-infrared absorbance above 1100 nm and is observed through pronounced absorbance bands. Protein in its various forms, and in particular collagen, is a strong absorber of light that irradiates the dermis. Near-infrared light that penetrates to subcutaneous tissue is absorbed primarily by fat. In the absence of scattering, the absorbance of near-infrared light due to a particular analyte, A, is approximated by Beers Law at each wavelength according to equation 1
A=εbC (1)
where ε is the analyte specific absorption coefficient, C is the concentration, and b is the pathlength. The overall absorbance at a particular wavelength is the sum of the individual absorbances of each particular analyte given by Beer's Law. The concentration of a particular analyte, such as glucose, is determined through multivariate analysis of the absorbance over a multiplicity of wavelengths because ε is unique for each analyte. However, in tissue compartments expected to contain glucose, the concentration of glucose is at least three orders of magnitude lower than that of water. Consequently, the signal targeted for detection by reported approaches to near-infrared measurement of glucose concentration, the absorbance due to glucose in the tissue, is expected to be at least three orders of magnitude lower than other interfering tissue constituents. Therefore, the near-infrared measurement of glucose concentration uses a high level of sensitivity over a broad wavelength range and the application of methods of multivariate analysis.

The spectral scattering characteristics of diffuse remittance from tissue are the result of a complex interplay of the intrinsic absorption and scattering properties of the tissue, the distribution of the heterogeneous scattering components, and the geometry of the points of irradiation relative to the points of light detection. Scattering in tissue results from discontinuities in refractive index on the microscopic level, such as the aqueous-lipid membrane interfaces between each tissue compartment or as the collagen fibrils within the extracellular matrix. The spatial distribution and intensity of scattered light depends upon the size and shape of the particles relative to the wavelength and upon the difference in refractive index between the medium and the constituent particles. The scattering of the dermis is dominated by the scattering from collagen fiber bundles in the 2.8 μm diameter range occupying twenty-one percent of the dermal volume and the refractive index mismatch is 1.38/1.35.

Dynamic Properties of Skin

At a given measurement site, both long and short term variation in the physiological state of tissue profoundly effect the optical absorbance and scattering properties of tissue layers and compartments over a relatively short period of time. Additional factors affecting tissue state include: temperature, hydration, applied pressure, relative thickness of skin layers, sampling position, localized absorbance coefficient, localized scattering coefficient, and anisotropy. In addition, such variations are often dominated by fluid compartment equalization through water shifts and are related to hydration levels and changes in blood analyte levels.

Sample Variation Compensation

The diverse scattering characteristics of skin cause light returning from an irradiated sample to vary in a highly nonlinear manner with respect to tissue analytes and in particular glucose. Simple linear models, such as Beer's Law, are invalid for analysis of highly scattering matrices, such as the dermis. This is a recognized problem and several reports have disclosed unique methods for compensating for the nonlinearity of the measurement while providing the necessary sensitivity.

K. Hazen, Glucose determination in biological matrices using near-infrared spectroscopy, Doctoral Dissertation, University of Iowa, (August 1995) and J. Burmeister In-vitro model for human noninvasive blood glucose measurements, Doctoral Dissertation, University of Iowa (December 1997) describe several methods of noninvasive glucose measurement that use calibration models that are specific to an individual over a short period of time. This approach avoids modeling the differences between patients and therefore does not generalize to more individuals. Further, the calibration models have not been tested over long time periods and do not provide means for compensating for the varying optical properties of the sample that occur over short time periods.

S. Malin and T. Ruchti, An intelligent system for blood analyte prediction, U.S. Pat. No. 6,280,381 (Aug. 28, 2001) describes a method for classifying spectra on the basis of structural and state similarity into clusters, such that the variation within a class is small compared to the variation between classes. Calibration models are developed for each developed cluster. The intelligent system design results in a plurality of models that is cumbersome in use.

Subject-Tailored Calibration Model

E. Thomas, R. Rowe, and M. Haass, Methods and apparatus for spectroscopic calibration model transfer, U.S. Pat. No. 6,441,388, issued Aug. 27, 2002, which is a continuation-in-part of E. Thomas, R. Rowe, Methods and apparatus for tailoring spectroscopic calibration models, U.S. Pat. No. 6,157,041 issued Dec. 5, 2000, (hereinafter the “'041” patent) and U.S. Pat. No. 6,528,809 issued Mar. 4, 2003, which is a continuation of the '041 patent describe a method and apparatus for noninvasively measuring a biological attribute using a subject-tailored calibration model. In the calibration phase, the calibration model data is modified to reduce subject-specific attributes resulting in a calibration data set modeling within-subject physiological variation, sample location, insertion variations, and instrument variation. In a prediction phase, the prediction process is tailored for each target separately using a minimal number of spectral measurements for each subject. This method uses approaches that include removal from subsequent spectra the first spectrum of a day or a mean spectrum. Mean-centering on the basis of a known external variable, such as those not related to the spectrum, is inherently limited due to physiological changes and other uncontrolled factors that occur over the time period of minutes, hours, and days. Such information is, at times, related to the signal of interest but is not necessarily a true and proper representation of it. Therefore, a new approach is necessary that is not limited by the inherent short-comings of external, supervised approach using a static adjustment technique in an environment continually resulting in observed absorbance and scattering variation within a subject.

### BRIEF SUMMARY OF THE INVENTION

Data varies as a result of chemical, physical, biological, and environmental changes. For example, in noninvasive glucose concentration estimation, noninvasive spectra vary due to a large number of parameters including at least:
(a) variation within an analyzer across time;
(b) variation between analyzers;
(c) variation between samples, such as subjects;
(d) spatial variation within a subject;
(e) temporal variation within a subject;
(f) fluid movement within a subject; and
(g) temperature change.

While knowledge and use of the optical properties of skin, high instrument sensitivity, and compensation for the inherent nonlinearities are vital for the application of near-infrared spectroscopy to noninvasive blood analyte measurement, an understanding of biological and chemical mechanisms that lead to time dependent changes in the optical properties of skin tissue is equally important and yet largely ignored. At a given measurement site, skin tissue is often assumed to be static except for changes in the target analyte and other absorbing species.

To date, no method exists for compensating for tissue related variation as described, supra. Particularly, no method exists that effectively compensate for highly nonlinear effects related to sampling different tissue locations; changes related to the complex, heterogeneous, multi-layered, and dynamic composition of tissue; the profound variation over time, from sample-to-sample and between patients; and the changes in optical properties related to the re-distribution of water between various tissue compartments. The fundamental assumptions of the current art, such as the constancy of multiplicative and additive effects across the spectral range, constancy of sampled pathlength, and homoscadasticity of noise are violated in the noninvasive tissue application. In particular, current calibration methods are inadequate for compensation for re-distribution of water between various tissue compartments that alter the optical properties of the tissue through changes in the water concentration, the concentration of other analytes, varying refractive indices of various layers, changes in the thickness of tissue layers, and the size and distribution of scattering centers, which result in the optical properties of the tissue sample varying in a highly nonlinear and profound manner.

The method of calibration would benefit from a method that attenuates the components of spectral interference related to the heterogeneity of the tissue, patient-to-patient differences, and variation through time, such as physiological effects. In view of the problems left unsolved by the prior art, there exists a need for a method and apparatus to reduce interference in tissue measurements related sample heterogeneity, time related variations, patient-to-patient differences, and instrument variation. Further, there exists a need to effectively compensate interferences from a variety of tissue types in the presence of environmental and instrument changes prior to calibration. Still further, there exists a need for the interference removal to be automated and exist in a readily applied fashion.

### SUMMARY OF THE INVENTION

The invention relates to a method and apparatus for calibration development using clustering. More particularly, the invention relates to subsequent calibration development using clusters that are individually interference compensated and to subsequent estimation of analyte property values from data, such as noninvasive spectra.

### BRIEF DESCRIPTION OF THE DRAWINGS

FIG. 1 is a functional schematic diagram of a calibration method according to the invention;

FIG. 2 is a schematic diagram of an architecture for a neural network system according to the invention; and

FIG. 3 is a flowchart of a method for data clustering, interference removal, and data aggregation prior to model development according to the invention.

### DETAILED DESCRIPTION OF THE INVENTION

A method and apparatus for development of a calibration model is described. More particularly, a method of developing and using a calibration model using clusters and interference removal in combination with aggregation of interference reduced data is described. Exemplar apparatus, analytes, and algorithms are provided using the example of analyte property estimation from noninvasive spectra.

Within the field of tissue analysis, noninvasive analyzers allow a multitude of analytes or structural features to be determined. The calibration principles apply to noninvasive measurements of a multitude of blood or tissue analyte properties. For example, based upon knowledge of the incident photons and detected photons, a chemical and/or structural basis of the sampled site is deduced.

Optionally, methods and apparatus are previously described in U.S. patent application Ser. No. 10/472,856 and U.S. provisional patent application No. 60/362,885, filed Mar. 8, 2002, both of which are incorporated herein in their entirety by this reference thereto.

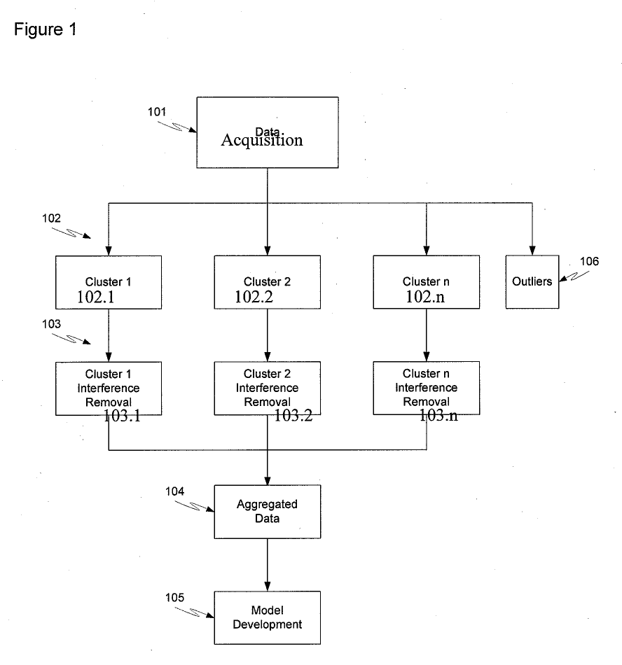

FIG. 1 is an overview functional schematic diagram of a method that includes processing data into one or more clusters, removing interference from one or more of the clusters, aggregating the data into a calibration data set, generating a model using the aggregated data, and implementing the model for analyte property estimation. Initially data, such as spectra, are collected (block 101). The data are classified (blocks 102) into one of n clusters 102.1, 102.2, . . . , 102.n, where n is a positive integer, based upon similarity. Preferably, the clustering is performed in an unsupervised manner, though supervised clustering is also usable. Data not falling within any cluster parameter are characterized as outliers 106. A basis set is developed, as described infra, for each cluster. An example of a basis set is a common interference within a given cluster. The determined interference, represented by the basis set, is then removed from the data (block 103). The resulting clusters 103.1, 103.2, . . . , 103.n, with interference removed, are then aggregated into a new data set (block 104). Optionally, bias for a given cluster is removed from the aggregated data or from one or more individual clusters (not shown). A calibration model is developed (block 105) using the resulting data. Subsequent prediction is performed on the prediction data, which is preferably processed through the same clustering/interference removal process based upon the reduction of interference using a basis set.

The prediction process involves assigning the prediction spectra to the most suitable cluster through a classification step, which is used to determine the cluster to which the spectra is closest. The classification is performed on extracted features using a distance metric, such as the Euclidean, Mahalanobis or absolute distance from either a representation of the cluster center, such as a mean, median, or average, or a set of cluster members, such as a k-nearest neighbors. Alternately, linear discriminant analysis or alternate classification analysis may be applied.

An optional outlier identification step may be employed to ensure that the prediction spectra or derived features possess a minimum distance from the selected class. Illustratively, the calculated distance metric may be compared to a maximum level; samples in excess to this point are rejected.

Prior to measurement, interference is removed from the prediction sample according to the predefined interference basis through a simple subtraction of a rotation which provides an orthogonal measurement relative to the interference. The optional prediction is set according to the bias associated with the interference basis set.

Interference Removal Model

In one illustrative interference removal model, the calibration accuracy of noninvasive analyte property estimation may be improved through the reduction of dominant sources of interference by the use of features extracted from spectral measurements to form clusters or sub-groups within a given calibration set. The features are used as a measure of spectral similarity or conversely, spectral distance and are used to group the data to produce clusters with similar interference patterns. A basis set is formed for each cluster defining the locally represented interference and the associated data are transformed to a common basis through the removal of the interference. With the interference removed, the data are aggregated into one set and used for the purpose of calibration model development and/or to estimate the concentration of an analyte in tissue through the application of a previously developed multivariable calibration model.

Clustering Model

In one illustrative clustering model, data is first clustered using an unsupervised approach, interference is removed from at least a plurality of clusters, a plurality of clusters are aggregated, and a model is developed using the aggregated interference removed data. Subsequently, a second data set is obtained, the second data set is processed in a manner tracking the data processing steps used to obtain the aggregated calibration data, and the model is applied to the second data set to yield an analyte property estimation.

This approach involves the collection of tissue measurements, the extraction of features representing interference, the unsupervised clustering of the data on the basis of the extracted features, the formation of an interference basis set for each cluster, the transformation of each cluster using the developed basis set upon each cluster, the aggregation of interference removed clusters, and the optional identification and removal of outliers.

Neural Network Model

In another approach, a nonlinear model is used for calibration and measurement. The model architecture is preferably defined by a network of simple interconnected nonlinear units called an artificial neural network. The network inputs include a measurement spectrum and one or more calibration spectra and corresponding reference values, such as calibration glucose measurements. Optionally, the network inputs include any of: the time of a measurement, the last series of network outputs, one or more subject characteristics, one or more sample subject spectra, and one or more environmental parameters. The network output is the estimated analyte property, such as a glucose concentration.

The nature of the neural network system is novel in that by using calibration points as input variables the relationship between the calibration points and subsequent measurements is modeled, thereby creating a more accurate and robust measurement amid variation in tissue scattering properties.

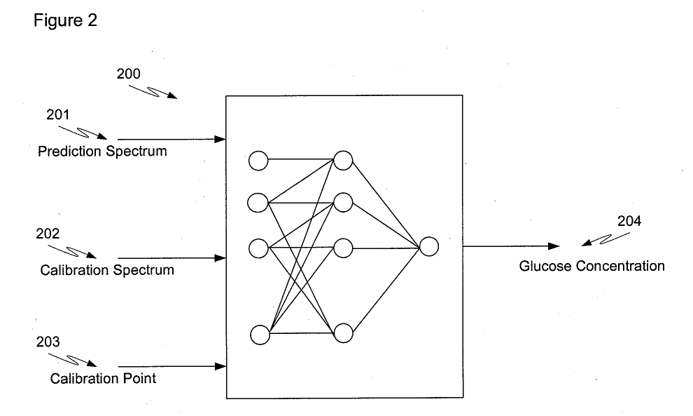

FIG. 2 is a schematic diagram of an architecture for a neural network system 200. For example, the nonlinear noninvasive near-infrared glucose determination system is nonlinear. The neural network has an input for calibration spectra 201 and for a prediction spectrum 202. Alternately, the spectral inputs are processed, re-sampled, filtered, scaled, normalized and/or decomposed prior to application to the neural network. However, the neural network has an additional calibration input also known as a calibration point 203. An example of an input is a spectrum from the individual that the network is predicting on, such as a spectrum from the individual collected on the day of the prediction spectrum. Additional input examples include an environmental effect and/or a bias. This system allows for use of information from a subject being tested for glucose concentration while leaving the neural network model unadjusted. Further, the unadjusted nonlinear neural network model provides a more robust estimate of an analyte concentration 204, such as glucose concentration in nonlinear near-infrared spectra.

Preferably, the artificial neural network is a feed-forward system, which includes one input for each element of the measurement and calibration spectra and one input for the calibration point. However, a recurrent artificial neural network and/or additional variables may be employed in cases in which a time dependency exists between the calibration point and the measurement and is particularly useful in the case of continuous monitoring systems.

The development of the model is performed by selecting the architecture where the architecture includes: a number of layers, nodes per layer, nonlinearity in each node, and bias units. A training step is used with the model. During training paired point of input variables and reference output variables are provided to an algorithm, which estimates weights of the network. Weight estimation techniques include use of any of: a gradient descent, conjugate gradient descent, nonlinear recursive least-squares estimator, or an extended Kalman filter. In addition, the training algorithm optionally uses covariance modifications, such as variable forgetting factors; a genetic algorithm; and simulated annealing to enhance the search capabilities.

Using the neural network process, the artificial neural network is trained and not adjusted with additional data, such as spectra from an individual on a measurement day. The neural network is preferably used in a system having inner filter effects and/or in a nonlinear system. Thus, the neural network is application in a nonlinear system where local clustering is not the most appropriate method. Local clustering and the construction of linear models is applicable in situations in which the variance of the analytical signal assumes a linear behavior in the vector space spanned by the system of factors. However, this presumes a well defined and linear relationship between the tissue scattering properties and the spectral measurement. Unfortunately, physiological variation, including fluid movements caused by typical variation in glucose levels, induce changes in the scattering properties of skin, which may best be characterized by a nonlinear representation of the system. In addition, repeated measurement of the skin and natural fluid redistribution through time leads to changes in the visco-elastic properties of skin, which is characterized by a nonlinear behavior with respect to time, glucose concentration and device usage.

Consequently, methods that utilize a calibration point, where the calibration point includes a spectrum and associated glucose value, to locally center subsequent data enable the combination of data collected through time or group data collected from multiple subjects fail to account for a significant source of spectral variability. Inevitably, this leads to a degraded or biased measurement and is an inadequate approach under many circumstances, such as environmental changes in temperature. The neural network approach is thus preferentially used in a nonlinear system, such as noninvasive glucose concentration estimation using near-infrared light.

After the nonlinear model has been defined and parameterized or trained, a measurement is made relative to the calibration point, which is a spectrum and associated reference glucose concentration. The calibration point is preferably set once for all instruments, subjects, and samples. However, improved accuracy may be obtained if the calibration point is collected for each individual instrument and/or close to the time the measurement point is acquired. In addition, in the case of recurrent network applications, successive calibration points throughout a multiple-day measurement period enable a more accurate glucose prediction.

The measurement point involves the collection of a spectrum, an optional processing step and the application of the spectrum and calibration point to the artificial neural network. The output of the artificial neural network is a glucose estimate, which may require further scaling and/or bias adjustment.

Finally, the network architecture may be defined to include multiple outputs for additional tissue constituents and analytes, physiological conditions, such as low blood sugar and an error indicator.

Unsupervised Model

Another approach is a method for processing a calibration set that substantially reduces spectral interference. This approach relies on unsupervised pattern recognition to develop natural groupings of the data. External variables need not be used to group data prior to processing. Rather, the grouping or clustering is performed by considering variation that is manifested in the data most significant to the measurement process. As a result, clusters are arranged based on variation that is the most detrimental to the measurement system or based on spectral similarity. In addition, data that is inconsistent with the derived clusters is identified and removed, a step which further enhances the quality of the calibration set. Finally, the individual clusters are transformed through an internal basis set to eliminate the interference that commonly ties the associated samples together.

The strategy employed is to represent multivariate interference in the form of features. Samples with similar interference are grouped and a basis set is formed capturing the similarity. By localizing the variation, the interference is significantly attenuated or removed thereby enhancing the analyte signal of each cluster. During the interference removal process each cluster is transformed to a common basis and glucose is measured relative to the basis.

This approach leads to the attenuation of tissue variability, which is manifested in spectral measurements. During the process of calibration, the reduction in spectral interference leads to parsimonious and robust models that are applied to a broader range of different tissue types, characteristics, and conditions. As applied to noninvasive analyte property measurement, this approach results in a profound improvement in accuracy.

For example, the unsupervised model takes the natural structure of the data to define the class or cluster centroid, where the cluster centroid is defined in the spectral cluster space. The centroid or a sample close to the centroid is then used as the bias point or interference.

In another example, a supervised and unsupervised system are contrasted. If an expert chooses to use a first spectrum of a day for an individual, the method is supervised. This supervised approach does not use clustering information. Further, the supervised approach of selecting a first or any spectrum of individual for a given day is unlikely to represent the class centroid. Indeed, the supervised approach of using a library selection or a linear combination of spectra does not represent the class centroid. Still further, in the near-infrared spectroscopy of tissue, the supervised approach using linear combination spectra fails in estimation of a realistic centroid due to non-additivity of spectral components of the linear combination. In stark contrast, a natural selection of the class centroid is an unsupervised method. The inventors have recognized that the use of an unsupervised method where the natural structure of the data is used to determine a centroid or a bias point results in superior local centering of the calibration model. Particularly, the unsupervised approach of local centering minimizes the nonlinear span of the measured data in the calibration space resulting more robust linearized predictions.

In another approach, the following steps are used in calibration development:
process spectra to enhance features related to the optimal properties of tissue;
form clusters;
mean-center each cluster or project each cluster onto a basis set;
identify as outliers samples to far or distant from existing clusters or use the samples to form a new cluster for a subsequent adaptive calibration;
for a new set of data, if not close to a cluster, optionally form one or more new clusters;
mean center the y-values or reference values according to the same grouping.

This approach is based on the data rather than a blind separate and supervised criterion. The step of using a separate variable is eliminated. An internal criterion is applied to the data rather than an external grouping and it is supervised. Previously reported methods have used a supervised approach to local centering by defining a variable external to the measurement and grouping the data according to it. For example, the data is often grouped by subject and/or by the time period over which the data was collected. A flaw in this approach is that it is blind to the actual variation that is present in the data and will produce a sub-optimal localization of interference. In addition, there is no guarantee that the primary sources of spectral interference, such as tissue scattering changes due to the measurement process, pressure, shear stress or physiological effects, will be substantially reduced. If within a pre-defined group of data significant variation exists, the accessibility of the net analyte signal for the purpose of calibration is adversely affected resulting in a poor calibration. Alternately, it is possible to inefficiently group the data, which leads in the future to un-modeled dynamics.

An approach that overcomes the shortcomings inherent in supervised methods for grouping data uses unsupervised grouping in which the data itself is used to form clusters. No external variable, such as time period or subject is used and the data is centered according to the natural groupings that occur. In addition, the groupings are optionally formed from spectral features, which are representative of the elements of the variation that, when grouped, provide a consistent and representable set of data for calibration. A feature is a compact representation of a source of spectral variance related to a chemical structure or physical phenomenon of the tissue present in the spectra. Features are used in at least three manners. First, features are found that are not readily fit or modeled and are therefore damaging to the model system. Second, features are used to identify separation of clusters. Third, features are used to identify homogeneity within a cluster.

The following topics are described herein:
an instrument suitable for making a tissue measurement;
tissue measurement, the collection of a noninvasive tissue measurement using the instrument;
tissue sample;
basis set measurement, what a basis set is and how it is formed through tissue measurements;
basis set application, the transformation of tissue measurements through application of the basis set;
noninvasive analyte measurement, the use of transformed spectral measurements for calibration development of noninvasive analyte property determination; and
bias correction, the correction of the reference analyte value used for calibration and the bias adjustment of noninvasive analyte measurements.
   Data Collection and Processing

Cluster formation and processing techniques are described in terms of: the analyte, analyzer, algorithm, preprocessing, filtering, feature extraction, classification, basis sets, interference removal, projection, bias removal, data aggregation, calibration, prediction, and outlier detection. Further, without loss of generality, the specific application of the cluster formation and processing techniques to noninvasive glucose concentration estimation through near-infrared spectroscopy is considered.

Analyte

Generally, the analyte is any property measurable using the clustering algorithm described herein. For example, the algorithm applies to any data set having groupings, such as within the field of noninvasive analyte property estimation. Therein, the analyte is any constituent of skin tissue and/or blood or an analyte that tracks the concentration of a blood constituent. One particular analyte of interest is glucose. Other sample constituents of interest within skin and/or blood include but are not limited to: fats, such as triglycerides or forms of cholesterol; proteins, such as albumin or globulin; urea; bilirubin; and electrolytes, such as Na⁺, Ca²⁺, and K⁺ or various chelates or zwitterions.

Algorithm

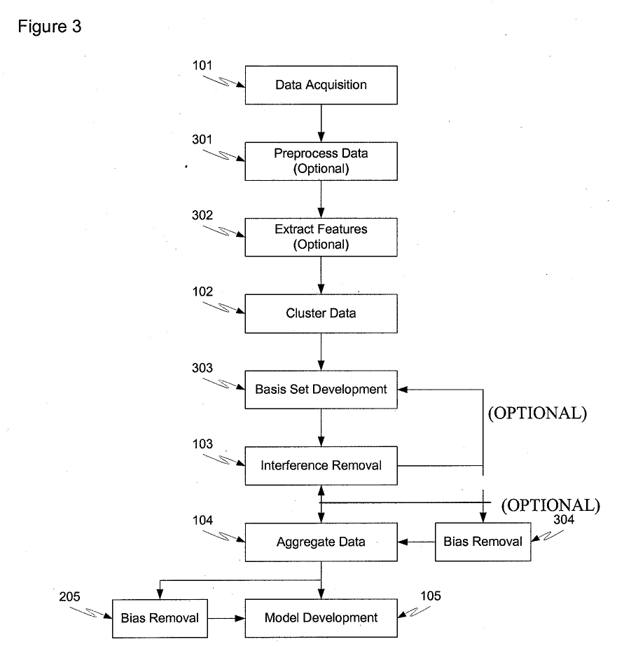

FIG. 3 is a flowchart of an illustrative algorithm. Initially, data are acquired (block 101). Optionally, the acquired data are preprocessed (block 301). A feature extraction step (block 302) is optionally performed on the preprocessed data or original data. The resulting data are subsequently clustered into one of n clusters (block 102), where n is a positive integer, based upon similarity. Data not falling with any cluster parameter are preferably characterized as outliers (block 106 in FIG. 1). A basis set is developed for at least one of the n clusters and preferably for each of the n clusters (block 303). An example of a basis set is a common interference within a given cluster. The determined interference is then removed from the data (block 103) for the corresponding cluster. Basis set development and interference removal are optionally repeated for the purpose of further reduction of interference. The resulting interference removed clusters are then aggregated into a new data set (block 104). Optionally, bias for a given cluster is removed from the aggregated data or from one or more individual clusters. A calibration model is developed (block 105) using the aggregated data. Optionally, bias is removed from the calibration model (block 205). Subsequent prediction is performed on data, preferably processed through the same cluster filters and interference removal process.

Data Acquisition (Block 101)

Using the example of noninvasive glucose concentration estimation, data or spectra are acquired using a near-infrared analyzer, such as those described in the commonly-assigned U.S. patent application Ser. No. 10/472,856, filed Mar. 7, 2003, which is incorporated herein by this reference thereto in its entirety as if fully set forth herein.

A noninvasive analyzer includes at least a source, detector, and algorithm. Optionally, an analyzer includes a spectroscopic measurement system, a sample interface module, and a computational analyzer. The spectroscopic measurement system detects the diffusely transmitted or reflected near-infrared radiation, within a specified range, from the targeted tissue, and corrects the measured spectrum for calibration and/or measurement of target biological parameters, properties, constituents, or analytes. The spectroscopic measurement system preferably includes subsystems for providing near-infrared radiation, selecting and/or controlling wavelength, interfacing to the sample for radiation delivery and recovery, analyzing the detected near-infrared radiation, and displaying the measured analyte, property, or constituent. The source radiates near-infrared energy, such as the wavelength range about 700 to 2500 nm and is, for example, an array of light emitting diodes (LEDs) or a broadband lamp, such as a halogen lamp. In the case of a broadband source, an optical filter is optionally used to reduce the effects of energy at wavelengths not in the spectral range of interest but that are emitted by the source of near-infrared energy. A method of wavelength separation before and/or after illumination of the sample is used with a broadband source. Examples of wavelength separation means include: a set of filters; a dispersive element, such as a plane, concave, ruled, or holographic grating; and an interferometer. Alternative techniques include successive illumination of the elements of an LED array or a Hadamard transform spectrometer. The sensing element is one or more detectors, which are responsive to the targeted wavelengths.

Preprocessing (Block 301)

A diversity of signal, data, or pre-processing techniques are optionally used in combination with the basic algorithm described herein with the common goal of enhancing signal, reducing noise, improving accuracy, and/or enhancing precision.

A method for the noninvasive measurement of biological parameters, agents, chemicals, properties, and constituents or analytes, for example, attenuates or removes the signal related to spectrally manifested changes or interference in the sampled tissue while substantially passing the analyte signal of the target analyte. The method is preferably performed on spectroscopic data collected by the instrument before generating a calibration model and before prediction of an analyte property using subsequently generated data.

Preprocessing may be optimized to maximize the ratio of analyte signal-to-noise. Optimization may be done empirically, such as through modeling or simulation; or theoretically by imposing the characteristics of or applying directly the preprocessing steps upon the known net analyte signal and related noise. For example, the break frequency of a low-pass filter is optimized to pass the signal of the analyte while attenuating the high frequency noise components. In a second example, a high-filter or bandpass cut-off parameters pass signal while restricting low frequency noise or both high and low frequency noise components, respectively. In yet another example, the wavelength range of multivariate scatter correction is selected based upon the signal-to-noise ratio on a wavelength by wavelength basis. In still yet another example, in cases where the noise involves significant interference a background subtraction step using the net analyte signal is optimized to remove noise relative to signal.

Many diverse preprocessing methods to remove spectral variation related to the sample and instrument variation are suitable for use, including: multiplicative signal correction, standard normal variate transformation, piecewise multiplicative scatter correction, extended multiplicative signal correction, pathlength correction with chemical modeling, and optimized scaling.

In addition, a diversity of signal, data, or pre-processing techniques directed at signal or net analyte signal accessibility or enhancement may optionally be used. The net analyte signal refers to the portion of the spectral signal related to the target analyte that is orthogonal to the interference.

Filtering

An additional optional preprocessing step is filtering. Filtering, which is an enhancement method, step, or operation, is used to remove the low frequency or baseline variation, such as changes resulting from variation in the reduced scattering coefficient and/or surface reflectance. Filtering may be accomplished through the application of a band-pass filter across the wavelength axis of the measured spectrum, which performs two basic functions. First, the low frequency variation over the wavelength axis of the measured spectrum is attenuated through a high-pass filtering operation. The cut-off frequency or bandwidth of the high-pass operation is determined according to the level of low-frequency interference and the frequency content of the net analyte signal of the targeted analyte. The second function is a low-pass or smoothing operation in which noise is suppressed through the attenuation of higher frequencies. The break frequency of the low-pass filter is set according to the bandwidth of the spectrometer, the net analyte signal of the target analyte or constituent and the necessary signal-to-noise ratio required to make the measurement. The two functions are optionally performed simultaneously or one at a time in either order depending on the implementation of the band-pass filter. The methods used for performing this operation include infinite-impulse response (IIR) and finite-impulse response (FIR) band-pass filtering. In the preferred embodiment, a FIR filter is implemented according to equation 2

$${m}_{f , j} = {\sum }_{k = 1}^{P} {a}_{k} {m}_{j - k - \frac{( p - 1 )}{2}} \qquad ( 2 )$$

where m_{f,i} is the filtered spectrum at the jth measured wavelength, mⱼ is the measured spectrum at the jth wavelength, aₖ denotes the kth filter coefficient, and P is the length of the filter impulse response or filter window width. In the equation, the filter is non-causal and applied across the wavelength axis of the measured spectrum. The filter width, P, is assumed to be an odd number and the filter coefficients are determined according to the desired filter break frequencies and characteristics of the pass and stop-bands. For example, a Savitsky-Golay first derivative is used to determine the filter coefficients with a smoothing window selected based on the noise characteristics, spectral sampling interval, and instrument bandwidth. In one application of the process for the measurement of glucose, a 31 nm smoothing window was determined to be optimal although window sizes between 15 and 61 nm were also determined to be effective. Alternately, the FIR filter design and coefficient determination is performed through one of many existing techniques, such as truncation and tapering of a target infinite impulse response sequence.

In another example of the filtering operation, when the sampling interval is large, such as when the wavelength discretization of the spectrum is course and/or the signal-to-noise ratio is sufficient, only the high-pass filtering operation is performed. A special case of the FIR high-pass filter is used in the form of a first difference of the spectrum.

In yet another example, the measured spectrum is oversampled with respect to the wavelength axis and the low-pass bandwidth is set equal to the optical bandwidth of the spectrometer. However, the break frequency of the low-pass section is optionally determined through an analysis of the signal-to-noise ratio where the net analyte signal is the signal and the noise is the root-mean-square variation of the measured spectrum in the wavelength region used for measurement of the target analyte. As the low-pass bandwidth is reduced, the high frequency components of the noise are attenuated leading to a reduction in the noise. However, this process also attenuates high frequency components of the signal leading to a simultaneous reduction in the net analyte signal. In an inventive alternative, in cases in which the spectrum is oversampled with respect to the wavelength axis, the noise is distributed in greater proportions at higher frequencies than the net analyte signal. Therefore, low-pass filtering the measured spectrum removes a greater proportion of the noise than the net analyte signal. The optimal low-pass bandwidth is defined as one that maximizes or increases the ratio of the net analyte signal to the noise. Given a frequency domain model including the net analyte signal, NAS(f), and the noise, N(f): the bandwidth of the low-pass filter, f_{bw}, is determined through equation 3

$${SNR ( {f}_{bw} )}^{2} = \frac{{\sum }_{f = 0}^{{f}_{bw}} {\vert NAS ( f ) \vert}^{2}}{{\sum }_{f = 0}^{{f}_{bw}} {\vert N ( f ) \vert}^{2}} \qquad ( 3 )$$

as the value for f_{bw} at which SNR(f_{bw}) is maximized. Alternately, this process is performed iteratively by filtering a wavelength domain representation of the net analyte signal and noise at various low-pass bandwidths or through an empirical set of data by selecting the break frequency to optimize the standard error of prediction or other figure of merit.

The break frequency of the high-pass section is set to attenuate low frequency variation caused by changes in the scattering while passing the net analyte signal. This is generally accomplished empirically through an exemplary set of data or through a harmonic analysis of the net analyte signal.

In still another example, one or more of the following operations may be performed to process the spectra:
averaging spectra;
correcting dead pixels;
calculating absorbance;
performing x-axis standardization;
uniformly re-sampling the spectrum to standardize the x-axis;
performing a first (gross) outlier detection;
correcting the spectrum;
performing a wavelength selection;
removing interference; and
performing a second (fine) outlier detection

The order of the operations is optionally varied to a limited degree. For example, the wavelength selection operation is optionally performed out of sequence, such as after the second outlier detection or before any of the earlier operations. In addition, not all steps are required. For example, correcting dead pixels is not appropriate to some analyzers. As a second example, conversion to absorbance is not always required.

Data preprocessing (block 301) is preferably performed prior to feature extraction (block 302). However, preprocessing (block 301) is optionally performed after feature extraction (block 302) or before and after feature extraction (block 302).

Feature Extraction (Block 302)

Optionally, feature extraction (block 302) may operate on the acquired data 101 or preprocessed data. Feature extraction is any mathematical transformation that enhances a quality or aspect of the sample measurement for interpretation. The general purpose of feature extraction is to concisely represent or enhance any of the structural, chemical, physiological, and optical properties of the tissue measurement site that are directly or indirectly related to the target analyte. For the purposes of the method of FIG. 3, a set of features is developed that is indicative of the effect of the target analyte on the probed tissue. The set of features represents or reflects tissue properties or characteristics that change in various ways according to changes in any of the structural, chemical, physical, and physiological state of the tissue. The changes in tissue state, in turn, are themselves directly or indirectly related to the target analyte.

Direct/Indirect

In this context, a direct measurement is defined as a measurement based on the signal generated by the analyte during the measurement process. An indirect measurement is based upon a physical or chemical property or characteristic that is correlated to the target analyte; but in the indirect measurement the analyte is not the direct source of the measured signal. For example, a direct glucose determination may be based upon any of the analyte absorbance bands, such as glucose absorbance bands at approximately 1590, 1730, 2150, and 2272 nm. The glucose absorbance bands are due to C—H and O—H bonds. An indirect glucose determination can be based upon absorbance bands. Exemplar absorbance bands in noninvasive spectra include water, protein, fat, and glucose absorbance bands. Water absorbance bands centered at approximately 1450, 1900, or 2600 nm. Similarly, an indirect measurement can be based upon absorbance bands centered at approximately 1675, 1715, 1760, 2130, 2250, or 2320 nm for fat or approximately 1180, 1280, 1690, 1730, 2170, or 2285 nm for protein. Another form of indirect measurement would be based upon scattering of light. In the example of noninvasive measurement of glucose through near-infrared spectroscopy, current approaches use the absorption of light due to the glucose molecules present in the sampled tissue volume to make a glucose determination. Conventionally, feature extraction is based on the absorbance due to glucose that can be uniquely identified from the background interference.

Indirect methods of measuring glucose include the use of factors that are affected by the concentration of glucose, such as the fluid distribution in the various tissue compartments. Other terms for an indirect reading include: physiologically correlated, correlated response, secondary response, secondary mechanism, glucose induced response, or analyte induced tissue response.

Advantageously, features are extracted that represent changes in the state, such as physical, chemical and physiological properties or characteristics, of the tissue from a prior state, distinct from the target analyte, in response to changes in the concentration of a target analyte, that occur as represented in the measured changes in tissue properties. For example, a change in glucose concentration triggers a redistribution or movement of fluids between extra-cellular, intra-cellular, extra-vascular, and intra-vascular compartments. The features targeted for extraction, therefore, may represent tissue properties related to any of:
a direct analytical signal;
an indirect analytical signal;
the concentration of water in each of the compartments;
the relative concentration of water in the compartments;
the size of the various compartments;
the change in electrical impedance resulting from the redistribution of water; and
the change in radiation emanating from the tissue.

Subsequently, extracted features are used or analyzed to identify conditions unsuitable for analyte measurement and/or to perform an actual measurement of a tissue analyte. For example, in the case of noninvasive measurement of glucose through near-infrared spectroscopy, a resolved estimate of the magnitude of the fat band absorbance is used to infer specific information about the dermis. Although fat is relatively absent from the dermis, near-infrared radiation must propagate through the dermis to penetrate the adipose tissue beneath. Thus, physiological changes lead to corresponding changes in the optical properties of the dermis that influence the level of near-infrared radiation that penetrates to and is absorbed by the fat in adipose tissue. Therefore, the magnitude of the fat band present in a near-infrared absorbance spectrum varies, in part, according to the variation in the optical properties of the dermis. For example, as the water concentration in the dermis increases, the detected magnitude of the fat band naturally decreases and vice-versa. Thus, the magnitude of the fat is a marker indicative of the analytical signal and/or pathlength.

Several types of features are optionally used for:
outlier detection;
compensation for changes in the properties of tissue; and
analyte measurement.

Given the tissue measurement, m (or the preprocessed measurement, x):
a simple feature is derived directly from the tissue measurement;
an additional feature, such as derived features, is determined from the simple features through one or more mathematical transformation such as addition, subtraction, division, and multiplication; and
an abstract feature is derived through linear and nonlinear transformations of the tissue measurement.

While simple and derived features generally have a physical interpretation related to the properties of the tissue, such as the magnitude of the fat absorbance, the set of abstract features does not necessarily have a specific interpretation related to the physical system. For example, the scores of a factor analysis, principal component analysis, or partial-least squares decomposition are used as features, although they have no direct physical interpretation or their physical interpretation is not always known. The utility of the principal component analysis is related to the nature of the tissue measurement. The most significant variation in the tissue measurement is not caused directly by glucose but is related to the state, structure, and composition of the measurement site. This variation is modeled by the primary principal components. Therefore, the leading principal components tend to represent variation related to the structural properties and physiological state of the tissue measurement site and, consequently, reflect the tissue properties.

Alternatively, the entire tissue measurement, after suitable preprocessing, is selected within the measurement module for development of a calibration model 105 via cluster identification 102 followed by basis set identification 303 and interference removal 103.

Feature extraction determines the salient characteristics of measurements that are relevant for clustering. A goal of clustering is to develop optimal sub-groups which have a maximized similarity of the extracted features. There are two operations involved, the first is the determination of a measure of similarity and the second is the assignment of sub-group measurement.

Class definition is performed through either a supervised or an unsupervised approach. In the supervised case, classes are defined through an external variable under the assumption that the variable is indicative of actual separation in the data. The use of external information in this manner is the first step in supervised pattern recognition which develops classification models when the class assignment is known. This is the method applied by several reported methods, such as in previously cited U.S. Pat. Nos. 6,441,388; 6,157,041; and 6,528,809. However, supervised class definition are not optimal due to the significant variation and nonlinearity of the data. The drawback of this approach is that attention is not given to the true level and nature of the spectral interference. For example, data grouped according to subject and localized by time provide a degree of reduced spectra variation. However, positional differences and environmental differences lead to significant variation on the order of the interference that was targeted for removal.

In stark contrast, unsupervised methods rely solely on the spectral measurements to explore and develop clusters or natural groupings of the data in feature space. Such an analysis optimizes the within cluster homogeneity and the between cluster separation. Clusters formed from features with physical meaning are interpreted based on the known underlying phenomenon causing variation in the feature space. Therefore, the approach has the significant advantages over the supervised methods and additionally eliminates the need for an external variable, such as the patient or data collection time period. Typically these methods involve feature selection and extraction; proximity, measurement, such as that based on similarity; and a clustering criterion

The statistical classification methods are applied to mutually exclusive classes whose variation is preferably described statistically. Once class definitions have been assigned to a set of exemplary samples, the classifier is designed by determining an optimal mapping or transformation from the feature space to a class estimate which minimizes the number of misclassifications. The form of the mapping varies by method as does the definition of optimal. Existing methods include linear discriminant analysis, soft independent modeling of class analogies (SIMCA), k nearest-neighbor and various forms of artificial neural networks. The result is a function or algorithm that maps the feature to a class, c, according to equation 4
c=f(z) (4)
where c is an integer on the interval [1,P] and P is the number of classes. The class is used to select or adapt the calibration model, as discussed supra.
Fuzzy Classification

While statistically based class definitions provide a set of classes applicable to blood analyte estimation, the optical properties of the tissue sample resulting in spectral variation change over a continuum of values. Therefore, the natural variation of tissue thickness, hydration levels, and body fat content, among others, results in class overlap. Distinct class boundaries do not exist and many measurements are likely to fall between classes and have a statistically equal chance of membership in any of several classes. Therefore, hard class boundaries and mutually exclusive membership functions appear contrary to the nature of the target population. A more appropriate method of class assignment is based on fuzzy set theory Zadeh, L. A. “Fuzzy Sets,” Inform. Control, vol. 8, pp. 338-353, 1965, which hereby is incorporated herein in its entirety by reference thereto.

Generally, membership in fuzzy sets is defined by a continuum of grades and a set of membership functions that map the feature space into the interval [0,1] for each class. The assigned membership grade represents the degree of class membership with “1” corresponding to the highest degree. Therefore, a sample can simultaneously be a member of more than one class.

The mapping from feature space to a vector of class memberships is given by equation 5
cₖ=fₖ(z) (5)
where k=1, 2, . . . P, fₖ(•) is the membership function of the kth class, cₖε[0,1] for all k and the vector cε [symbol] ^{P} is the set of class memberships. The membership vector provides the degree of membership in each of the predefined classes and is passed to the calibration algorithm.

The design of membership functions uses fuzzy class definitions similar to the methods previously described. Fuzzy cluster analysis is optionally applied and several methods, differing according to structure and optimization approach are optionally used to develop the fuzzy classifier. All methods attempt to minimize the estimation error of the class membership over a population of samples.

Given the set of features, measures of spectral similarity are subsequently calculated, for example using distance metrics. In addition, the number of developed groups optionally vary depending on the method and the strategy employed. In one implemented method k-means clustering is performed with optimal group determination. Using this approach, the features are transformed according the their Mahalanobis distance and groups are formed by optimizing the homogeneity, such as minimizing the within group distance.

Hierarchical clustering approaches, such as use of a classification and regression tree (CART), are alternatively. For example, two extracted features associated with the fat and protein bands of the first overtone are used in the clustering analysis. Alternatively, data is clustered with overlapping clusters. This may be performed using k-nearest neighbors analysis, which develops a distinct cluster for each sample that overlap significantly and is effective for the removal of interference.

Basis Set Development (Block 303)

A basis set is developed (block 303) for a given cluster 102. A basis set is a set of one or more fundamental spectra capable of representing a constituent or interference of a sample matrix. Optionally, a basis set is developed as in linear algebra where a basis is a minimum set of vectors that, when combined, can address every vector in a given space. In a first example, a basis set represents a common interference within the spectra of a given cluster. In a second example, a basis set spans the vector space meaning that a linear combination of all the vectors yields every vector in the space. A basis preferably contains vectors that are linearly independent. Linear independence means that the solution to a homogeneous linear combination of the set has only a trivial solution; otherwise some of vectors could be formed from a linear combination of the other vectors in the set.

In the ideal case, the basis set spans the interference present in the data while remaining orthogonal to the signal of interest. Practically, however, the function of a cluster specific basis set is to attenuate gross interference that would otherwise prohibit the use of a diverse set of data for the purpose of calibration. According to the invention, the basis set is an estimate of the optimal, developed on the basis of empirical measurements.

A given cluster optionally represents raw data (block 101), preprocessed data (block 301), and/or extracted features (block 302). Hence, the basis set for a given cluster is developed using any of raw data, preprocessed data, and/or extracted features. Further, there exist a large number of methods of determining a basis set of a cluster, such as a common interference within a cluster, including determining for a given cluster any of:
a mean;
a centroid;
a median;
a weighted average of cluster representations;
a weighted average of the spectra within a cluster;
a first n factors for a cluster, where n is a positive integer and factors are determined using multivariate regression;
a tissue template;
any robust estimate of the mean of a cluster; and
a similarity measure.

A suitable method for enhancing the net analyte signal related to a particular analyte by transforming the corresponding spectroscopic measurement according to a basis set is described in S. Malin and K. Hazen, Method and Apparatus for Generating Basis Sets for Use in Spectroscopic Analysis, U.S. Pat. No. 6,11,673, Sep. 5, 2000, which hereby is incorporated herein in its entirety by this reference thereto. The '673 patent describes an alternative basis set as a spectral representation of at least one component found in a sample that is typically a source of interference. However, as described herein, the spectral measurement is transformed by the removal of the signal related to the basis set from the spectral measurement through removal means, such as subtraction, deconvolution, or rotation.

Basis Set Measurement

A tissue basis set, denoted by Sε [symbol] ^{P×N}, is a set of P vectors that represents components of interference present in a tissue sample. A basis set is formed through the collection of tissue measurements, mε [symbol] ^{1×N}, at various times and tissue locations under diverse conditions. In one example, application sources of interferences include any of:
tissue heterogeneity, such as sampling location;
structural and compositional differences patient-to-patient;
time dependent sources of interference, such as physiological variation; and
instrument variation, such as instrument-to-instrument differences and instrument variation through time.

A different tissue basis set is preferably generated for each cluster and represents the interfering background signal related to the overall optical properties of the tissue measurements. For example, the basis set represents the mean value of the cluster, a weighted mean, or a robust estimate of the mean. For example, it may be advantageous to use the cluster center as the basis. In these circumstances the basis set is a single spectrum. However, the basis set optionally includes additional spectra representing destructive interference common to all spectra.

After the determination of the basis set, the data are optionally preprocessed. It is beneficial to preprocess the basis set to attenuate random noise, baseline variation associated with the instrument, variation related to surface contact and low frequency interference related to scattering. Preprocessing steps include: filtering, averaging, derivative calculations, multiplicative scatter correction, smoothing, and/or normalization. The basis set is applied to transform preprocessed tissue measurements, x, to produce a corrected measurement, z. Therefore, it is preferable that the methods and steps used to preprocess the basis set be identical to those applied in the preprocessing step to tissue measurements.

In addition, in certain applications it is desirable to optimize the selection of tissue measurements used to create a basis set. The purpose for selecting an optimal subset of samples is to capture the characteristic background to which the primary energy absorbing and scattering constituents in the tissue contribute. The inclusion of samples with slight spectral variations not related to these tissue constituents results in the computation of an unrepresentative basis set and leads to a less efficient correction of the data. Four representative methods are described for performing sample selection prior to the determination of a basis set.

A first exemplary method is to compute a robust estimate of the mean of the data set targeted for the basis set. For example, a the trimmed mean is calculated by excluding the highest and lowest 25% of values at each wavelength or variable prior to averaging.

A second exemplary method is to perform a Principal Component Analysis (PCA) and to remove samples that contain high leverage with respect to the sample population. Several methods are optionally employed using PCA such as a leave-one-out analysis of the captured covariance from the resulting PCA eigenvalues. Samples that when left out result in a drop in covariance greater than a preset limit are preferably removed. In an alternate embodiment a T-Squared or Q-Test of the Principal Component scores is performed. Samples exceeding a defined confidence interval are preferably excluded from the basis set computation.

A third exemplary method for selecting a subset of samples is to process known spectral features into quantifiable information that is used to determine the state of the tissue encountered. For example, spectral bands containing information related to fat, water, protein, surface reflectance, probe-to-surface contact, and the like are compressed into single property values through processing and then used individually or in combinations through linear or complex functionality to determine samples that have information most consistent with the current optical state of the tissue. Samples associated with inconsistent optical states with respect to the calibration set or property values exceeding those predefined through calibration are preferably excluded. The remaining samples are used to compute the basis set.

A fourth exemplary method involves propagating the collected spectral measurements through a rudimentary predictive model and comparing the resulting analyte estimates to spectral features that are related to key optical characteristics of the encountered tissue. Measurements that have a high correlation to extracted features related to sampling anomalies, such as surface reflectance, are preferably excluded from the sample population. The remaining samples are used to compute the basis set.

Interference Removal (Block 103)

Interference removal removes or minimizes a common interference of a cluster or group. Interference removal reduces variation in the measurement, such as variation associated with sample-site differences, dynamic tissue changes, and subject-to-subject variation. Exemplar interference removal steps are described, infra.

The tissue measurement is preferably applied to the basis set through a transformation and a set of normalization parameters according to equation 6
z=f(x,S,P) (6)
where z is the transformed spectral measurement, S is the basis set and P is the set of weights or normalization parameters. The transformation, f(•), is a function that is used to attenuate the interference represented by S that is contained in x. The methods used for transformation include any of: subtraction, weighted subtraction, division, deconvolution, multiplicative scatter correction, and rotation.

Illustratively, the transformation may occur through equation 7
z=x−(c^{T}S+d) (7)
where cε [symbol] ^{1×P} is used to weight each member of the tissue basis set to optimally reduce the interference in x and dε [symbol] ^{1×N} is an intercept adjustment. The coefficients c and d are either preset or determined through multiple linear regression. An extension of this technique occurs when one tissue sample site is used. In this case, the basis set includes one processed tissue measurement associated with a particular time and guide placement and the basis set is applied to the processed tissue measurement through equation 8
z=x−S. (8)

For example, the attenuation or removal of interfering spectral variation is beneficial for the enhancement of the signal-to-noise ratio and the accurate and precise measurement of glucose.

First, a data set for calibration including: x_{c}ε [symbol] ^{P×N} and y_{c}ε [symbol] ^{P×1} where x_{c} is the matrix of tissue measurements and y_{c} is a vector of analyte concentrations. Further, x_{c} and y_{c} are collected or associated with a particular state. A state is defined by one or more of the following: subject, day, time, tissue position, temperature, environmental condition, tissue properties, etc. During calibration it is often advantageous to combine data associated with multiple states so that a more certain estimate of the analyte signal is determined. However, the different states often result in highly nonlinear variation that is difficult to model. Previously, we have subtracted the mean of x_{c} from x_{c} and the mean of y_{c} from y_{c} prior to calculating the regression vector. Now we have developed a novel method with great advantages. This method is to orthogonalize x_{c} to a set of background spectra, a basis set, which define or represent at least a portion of the interference. These we call x_{b}. x_{b} may be a set of rapidly collected measurements associated with a given state or combination of x_{c} such as the mean or the individual means. x_{c} is then processed as follows:
x′_{c}=x_{c}└I−x_{b}^{T}(x_{b}^{T})⁻¹┘ (9)
where (x_{b}^{T})⁻¹ is the pseudo inverse of x_{b}^{T},

$${y}_{c}^{′} = {y}_{c} - \frac{1}{L} {\sum }_{k = 1}^{L} {y}_{c , k} \qquad ( 10 )$$

where y_{c,k} is the k^{th} element of y_{c} and L is the set of spectra used to calculated x_{b}. The method provides a different and novel alternative that is superior to the standard practice of mean-centering the calibration set of mean-centering the calibration set according to locally defined means.

An additional method is recorded for tailoring an existing calibration to a particular cluster. Give a calibration defined by Wε [symbol] ^{1×N}, which is derived from one or more aggregated clusters, and a method of preprocessing, g(•): [symbol] ^{M} [symbol] [symbol] ^{N}, it is advantageous to tailor W to a cluster that is not necessarily contained, in terms of interference related variation, in the calibration set. That is, the calibration is preferably modified prior to application to data with un-modeled variation. The method is as follows
1. Represent the un-modeled variation via measurements, x
2. Process x=g(x)—note that this step is optional since in certain circumstances the use of processing is not necessary.
3. Calculate a new calibration by projecting the W onto the null space of the interference using
   W_{new}=└I−x_{B}^{T}(x_{B}^{T})⁻¹┘W (11)

A regression vector W is an estimate of the net analyte signal on the basis of a calibration set of exemplary data. However, the estimate is highly limited by the calibration set. If the calibration set does not suitably represent the variation associated with the interference then the measurement of the target analyte will be influenced and corrupted by the unmodeled variation. In this situation one of two methods may be employed. First, the calibration can by updated to properly compensate for the newly observed interference. Second, the measurements can be processed to remove the added interference. In both cases the optimal solution (in the linear sense) occurs through the orthogonalization of the regression vector or the newly acquired sample with the newly observed and representative set of interference.

As the spectra data measurements are transformed, they vary in a manner relative to a new basis set. Therefore, the associated reference property values are transformed to reflect this transformation. The new basis set is preferably set to a glucose value of zero by subtracting the reference property value associated with it. Similarly, the glucose value of the basis set is preferably subtracted from the property value of each individual sample. The resulting calibration set has variation that is substantially decreased.

Projection

In reference to the method of removing interference from calibration or measurement clusters, one method of interference removal for a set of data represented by a cluster is the use of a projection algorithm that projects each sample onto the null space of the interference, thereby removing the signal present in each sample that is related to the interference spanned by the related basis set.

The method is based upon attenuating interference modeled by a particular cluster's basis set by projecting each individual measurement onto the null space of the respective basis set or the space not spanned by the basis set. The result is the determination of the portion of the measured signal that is available for either calibration or measurement. The method also reduces the interference common to a particular cluster thereby enabling the aggregation of multiple clusters subsequent to the projection calculation.

In terms of robustness and efficiency, the projection algorithm removes interference more completely than subtraction and, unlike the latter, is the optimal solution for the linear case. A problem with methods based on the simple subtraction of a mean-spectrum is that interference is present in x′. That is, a portion of the interference may be removed but a simple subtraction does not necessarily yield a processed spectrum that is orthogonal to the representing interfering spectra.

The part of the measurement, xε [symbol] ^{1×N}, that is orthogonal to the M spectra of interferences represent in the matrix x_{b}ε [symbol] ^{M×N} is computed by
x′=└I−x_{b}^{T}(x_{b}^{T})⁺┘x (12)
where x′ is the projected spectrum that is orthogonal to the interference, N, is the number of variables associated with the measurement, such as distinct wavelengths, (•)⁺ is the pseudoinverse operator, and Iε [symbol] ^{N×N} is the identity matrix. The signal, x′, is the unique portion of the measurement x after the removal of the interference, through orthogonalization as represented in the set of spectra x_{b}. The quantity [I−x_{b}^{T}(x_{b}^{T})⁺] is the null space of the interference and is the basis set that spans the vector space which is orthogonal to x_{b}. The projection of x onto the null space of the interference is a transformation which reduces x to the essential signal, such that x variation that is similar to x_{b} is contained in x_{b}^{T}. Consequently, the potential influence of interference that is represented in the background interference is minimized.

Additional methods of interference removal include simple subtraction and adaptive filtering.

Bias Removal (Block 304 and Block 205)

Existing methods, such as multiplicative scatter correction and standard normal variate transformation, are used with the assumption that the multiplicative and additive sources of variation are uniform across the entire spectrum. However, in many applications, such as noninvasive measurement of glucose concentration, variation in the spectra in not corrected in this manner. Therefore, we describe an optional bias correction a step for correcting the non-linear variation resulting from sampling site differences that results from the heterogeneity and layered composition of the sample.

Background removal uses a basis set of spectral interferences to remove the signals that are specific to a given sampled tissue volume, the background. The optical estimate of the background is preferably performed subsequent to the removal of noise and the correction of the spectrum. If this operation is implemented prior to spectral correction, detrimental signal components remain in the spectrum that compromise the estimate of the background and lead to degraded results.

Background removal preferably follows the steps defined above and uses a spectral background or tissue template. For example, background removal may be performed by calculating the difference between the estimated spectral background or tissue template and x through
z=x−(cxₜ+d) (13)
where xₜ is the estimated background or tissue template, c and d are slope and intercept adjustments to the tissue template. Direct subtraction is just one form of background removal. The spectrally corrected signal, z, is used for calibration development or measurement of a target analyte. The background is estimated on the basis of an optimal selection of spectrally corrected measurements collected prior to the measurement, m. The variables c and d are preferably determined on the basis of features related to the dynamic variation of the tissue.
Aggregate Data (Block 104)

Subsequent to clustering (block 102) and interference removal (block 103), a plurality or all of the resulting clusters are aggregated. For example, matrices of response signal for each wavelength are appended together.

Model Development (Block 105)

Calibration or model development 105 is performed using the aggregated data 104. In the example of noninvasive glucose concentration estimation using near-infrared light, a calibration data set includes exemplary paired data points from one or more subjects collected over a period of time. Each paired data point includes a spectroscopic measurement, or spectrum, and a corresponding reference value for the analyte of interest. The calibration is a mathematical model, equation, or curve is developed on the basis of the calibration set and is used to subsequently determine the value of the analyte on the basis of a spectroscopic measurement. The invented method of spectral correction has been found to be beneficial for correction of both the spectroscopic data used for calibration and for subsequent measurement. Examples of models include those using principal component regression (PCR), weight PCR, partial least squares, artificial neural networks, multiple linear regression, and the like.

Prediction

Prediction or estimation of an analyte property, such as glucose concentration, is performed using the calibration model on a second data set, such as subsequently collected noninvasive spectra. Several prediction approaches are described here.

In a first prediction approach, a prediction spectrum is matched to one of the calibration clusters 102 based upon similarity. The interference 103 removed from the corresponding calibration cluster is removed from the prediction spectrum. The model is subsequently applied to the interference removed prediction spectrum to yield a predicted or estimated analyte property, such as a glucose concentration. In a second prediction approach, an interference is estimated for the particular prediction spectrum and the interference is removed with a projection algorithm prior to application of the model. In a third prediction approach, the calibration model is applied directly to the prediction spectrum to yield an analyte property estimation.

Outlier Detection

Optionally, outlier detection may be used in conjunction with the calibration or prediction use of the invention. The initial outlier detection operation removes aberrant spectra that 1) is not easily detected if performed after filtering and 2) is highly detrimental to the correction step magnitude calculation. Gross error detection executed in the steps immediately following the measurement of a spectrum is performed on the basis of specifications common to all samples and involves rudimentary tests for data acceptability. The tests for acceptability are made on the basis of specifications for noninvasive glucose measurement. If a deviation from the specified level of acceptability is detected the resulting action is the rejection of the collected spectrum, the rejection of the entire sample, or the generation of an instrument malfunction error.

Outlier detection is significantly enhanced by examining the net analyte signal after the removal of the interference. If the processed spectrum is significantly distorted, distant, or dissimilar from the calibration set, it is highly likely that the spectrum contains un-modeled variation that leads to uncertainty in the measurement. This metric is given by

$$z = {\sum }_{k = 1}^{N} {{a}_{k} ( {x}_{k}^{′} )}^{2} \qquad ( 14 )$$

where aₖ is a scaling factor for the k^{th} wavelength and is set according to the distant metric being used. If z exceeds a present limit defined according to the variation observed in the calibration set the sample is produces an analyte measurement with a low degree of certainty and is considered an outlier. The value of the method relies on the use of cluster centers to produce a local estimate of the net analyte signal. Simple methods of subtracting the mean, lead to a measurement that is biased high and lack the sensitivity useful for thorough outlier analysis.
Analyzer

The algorithm described herein may be used in a noninvasive analyzer, such as a glucose concentration analyzer, which has at least a source, a sample interface, and at least one detector. Optional analyzer components include at least: a backreflector, guiding optics, lenses, filters, mirrors, and a wavelength separation device.

Examples of noninvasive analyzers include:
a handheld device;
a tabletop device;
a device mounted onto a medical rack system;
a split module device, where the analyzer include a sample module separated from a base module
   a split module device coupling the base and sample modules with a communication bundle;
   a split module device where the base module and sample module are coupled via telemetry;
a device collapsible into a carrying case;
a tabletop device with a support module allowing weight support for a top-down instrument to patient interface; and
a device collapsible into a carrying case.

In one example, the analyzer folds into a carrying case. For example, the case is opened to access instrument controls and view screen and closed during transport. The closed analyzer optionally includes a handle or carrying strap. Preferably, the outside of the analyzer comprises outer surfaces of the carrying case.

In a second example, the majority of the mass of the analyzer is supported with a support module allowing the sample probe interface to minimize or essentially eliminate downward forces applied by the weight of the analyzer on a tissue sample site, thereby minimizing stress and strain at and/or about the sample site. The algorithm may be installed in the support module.

Additional optional components and/or controls of the apparatus include any of:
a targeting system;
an adaptive sample probe head;
a dynamic sampling probe;
a specular reflectance blocker;
occlusion and/or tissue hydration control;
a automated coupling fluid delivery system;
a coupling fluid temperature control system;
an automated coupling fluid delivery system;
a guide;
a mount;
a system for reducing stress/strain on the tissue;
a system for controlling skin tissue state;
a system for reducing and/or controlling thermal changes of the skin tissue;
an intelligent system for data processing;
a basis set; and/or
an embedded data processing algorithm.
   Dynamic Sampling Probe

A sample probe or sample probe tip of the analyzer is optionally used in a dynamic manner. For example, a targeting system sample probe and/or a measuring system sample probe of the analyzer are optionally dynamic. A dynamic probe is moved in a controlled fashion relative to a tissue sample in order to control spectral variations resulting from the sample probe displacement of the tissue sample during a sampling process.

For example, a noninvasive analyzer controls movement of a dynamic sample probe along any of the x-, y-, and z-axes and optionally controls tilt and/or rotation of the sample probe relative to a sampled tissue. In one case, a sample probe is controlled at least along the z-axis perpendicular to the x,y plane tangential to the surface of the sampled site thereby controlling displacement of the sample probe relative to a sample. The z-axis control of the displaced sample probe element of the sample module provides for collection of noninvasive spectra with proximate contact of the sample probe tip with the tissue sample, with a given displacement of a tissue sample, and for collection of noninvasive spectra with varying applied displacement positions of the sample probe relative to the nominal plane of the sample tissue surface.

Tissue Stabilizer

A tissue stabilizer is an interface element, where the element contacts the skin about the sample site of a subject. Preferably, the tissue stabilizer circumferentially surrounds the sample site, such as with a contacting ring or ellipse. Preferably, the interface element touches the skin at least a half-inch and preferably one inch or more away from the optically sampled tissue. The tissue stabilizer minimizes skin movement at the sample site. For instance, the tissue stabilizer reduces observed breathing responses in the spectral data. The stabilizer further minimizes applied stress/strain at and/or about the sample site. The stabilizer is preferably not attached to the sample site, but is placed into contact with the sample site just prior to or in conjunction with each tissue measurement. The tissue stabilizer is either integrated into the analyzer, replaceably attaches to the analyzer, or is separate from the analyzer. In one case, the stabilizer contacts the skin further away from the sample site along the axis of a limb or sample body part and contacts the skin closer to the sample site across the axis of the limb. In another case, the stabilizer is curved to approximate the curvature about the sample site. In still another case, the outer surface of a sample probe is greater than about one-half of an inch from the regions of the tissue stabilizer in contact with the human subject. Alternatively, the tissue stabilizer contacts the skin through three or more support structures, such as posts.

Those skilled in the art will recognize from the description set forth herein that the various embodiments of the present invention as described herein are illustrative, and that the present invention may be manifested in a variety of forms in addition to the specific embodiments described and contemplated herein. Departures from the embodiments in form and detail may be made without departing from the spirit and scope of the present invention. Accordingly, the invention should only be limited by the Claims included below.

1. A method for generating a model for noninvasive estimation of an analyte property of blood or tissue from spectral data, comprising the steps of:
clustering the spectral data into a plurality of clusters;
removing interference from each of said clusters to yield respective interference-reduced clusters;
aggregating said interference-reduced clusters into a calibration data set; and
generating said model from said calibration data set.

2. The method of claim 1, wherein said step of clustering comprises the step of unsupervised clustering.

3. The method of claim 2, wherein the clustering step comprises the step of establishing the clusters with a cluster spectral variation within the clusters that is less than spectral variation between said clusters.

4. The method of claim 3, wherein the clustering step comprises the step of clustering the spectral data into a plurality of clusters in accordance with predetermined parameters, and further comprising the step of:
identifying as an outlier spectral data which fails said predetermined parameters.

5. The method of claim 3, further comprising the step of:
developing a basis set for at least one of said clusters, wherein said basis set represents a common interference within said one cluster.

6. The method of claim 5, wherein said step of interference removing further comprises the step of applying a basis set to at least one of said clusters.

7. The method of claim 3, further comprising the step of:
implementing said model to estimate said analyte property.

8. The method of claim 7, further comprising the step of:
collecting a noninvasive spectrum, wherein said step of implementing further comprises the step of processing said noninvasive spectrum to yield said analyte property.

9. The method of claim 8, wherein said analyte property comprises a glucose concentration.

10. The method of claim 9, further comprising the step of:
prior to said step of implementing, removing interference from said noninvasive spectrum according to a predefined interference basis.

11. The method of claim 10, where said interference basis comprises a compact representation of a source of spectral variance within said spectral data related to a chemical structure or a physical phenomenon of the blood/tissue.

12. The method of claim 10, wherein said step of removing further comprises the step of subtracting a rotation, said rotation representing an orthogonal measurement relative to said interference basis.

13. The method of claim 8, further comprising the step of:
prior to said step of implementing, assigning said noninvasive spectrum to one of said at least two clusters through a classification step, wherein said classification determines closest proximity of said noninvasive spectrum to each of said at least two clusters.

14. The method of claim 13, wherein said classification uses a distance metric.

15. The method of claim 3, wherein said step of removing interference comprises the step of using a feature extracted from said spectral data in forming said at least two clusters.

16. The method of claim 15, wherein said feature comprises a compact representation of a source of spectral variance within said spectral data related to a chemical structure or a physical phenomenon of the blood/tissue.

17. The method of claim 16, further comprising the step of:
determining an outlier, wherein said outlier comprises a spectrum wherein said feature is not readily fit or modeled.

18. The method of claim 16, further comprising the step of:
separating said at least two clusters based upon said feature, wherein said feature provides a distinguishing characteristic between said at least two clusters.

19. The method of claim 16, further comprising the step of:
identifying homogeneity within a cluster based upon said feature.

20. The method of claim 1, further comprising the step of:
forming a basis set of locally represented interference for each of said at least two clusters, wherein said step of removing interference comprises the step of removing said represented interference.

21. The method of claim 2, wherein said unsupervised method comprises a nonlinear model.

22. The method of claim 21, wherein said nonlinear model comprises a neural network.

23. The method of claim 2, wherein said unsupervised method represents natural structure of said spectral data to define said interference.

24. The method of claim 2, wherein said unsupervised method reduces a nonlinear span of said spectral data in a calibration space.

25. The method of claim 2, wherein said unsupervised method updates independently of an expert system.

26. An apparatus for noninvasive analyte property estimation of a human subject having skin and a sample site, comprising:
a source of light energy;
a detector of light energy for acquiring spectral data;
a sample interface, the source and detector being disposed therein; and
an estimation component for estimating the analyte property from the spectral data, the estimation component comprising a calibration model component having an architecture of at least two clustered spectral data sets and means for removing separate cluster related interference from each of said clustered spectral data sets.

27. The apparatus of claim 26, further comprising:
a tissue stabilizer for reducing skin movement at the sample site.

28. The apparatus of claim 27, wherein said tissue stabilizer is adapted to contact the skin of the subject during use.

29. The apparatus of claim 28, wherein said tissue stabilizer is adapted to circumferentially surround and contact the sample site during use.

30. The apparatus of claim 29, wherein said tissue stabilizer is adapted to reduce stress and strain at the sample site.

31. The apparatus of claim 27, wherein said tissue stabilizer is adapted to contact the human subject no less than one-half inch from the sample site.

32. The apparatus of claim 31, wherein said tissue stabilizer is adapted to contact the human subject no less than one inch from the sample site.

33. The apparatus of claim 28, wherein said tissue stabilizer comprises a curved surface approximating curvature about the sample site.

34. The apparatus of claim 33, wherein said tissue stabilizer is adapted to contact the human subject no less than one-half inch from the sample site.

35. The apparatus of claim 33, wherein said tissue stabilizer is adapted to contact the human subject no less than one-half inch from the outer surface of a sample probe of said analyzer.

36. The apparatus of claim 35, wherein said analyzer is foldable into a carrying case.

37. The apparatus of claim 36, wherein at least one outer surface of said analyzer comprises at least one outer surface of said carrying case when said analyzer is in a folded state, wherein said carrying case further comprises a handle.

38. An apparatus for noninvasive analyte property estimation of a subject having skin and a sample site, comprising:
a source of light energy;
a detector of light energy for acquiring spectral data;
a sample interface, the source and detector being disposed therein; and
an estimation component comprising a processor and memory, the memory being coupled to the processor and comprising a plurality of processor instructions for:
controlling the acquisition of the spectral data;
clustering the spectral data into a plurality of clusters;
removing interference from each of the clusters to yield respective interference-reduced clusters;
aggregating the interference-reduced clusters into a calibration data set; and
generating a model for the analyte property estimation from the calibration data set.
